(19) United States
(12) Patent Application Publication
(10) Pub. No.: US 2025/0333792 A1
(43) Pub. Date: Oct. 30, 2025

(54) GENE PANEL FOR IDENTIFYING HYPO SEPSIS PHENOTYPE PATIENTS

(71) Applicant: University of Florida Research Foundation, Incorporated, Gainesville, FL (US)

(72) Inventors: Faheem Wagid Guirgis, Gainesville, FL (US); Srinivasa Reddy, Gainesville, FL (US)

(21) Appl. No.: 19/191,070

(22) Filed: Apr. 28, 2025

Related U.S. Application Data
(60) Provisional application No. 63/639,782, filed on Apr. 29, 2024.

(51) Int. Cl. C12Q 1/6883

(52) U.S. Cl. CPC C12Q 1/6883; C12Q 2600/106; C12Q 2600/118; C12Q 2600/158

(57) ABSTRACT

Described are methods of diagnosing or determining the risk of developing HYPO sepsis in a subject. Also disclosed are method diagnosis and treating or preventing HYPO sepsis in a subject. The methods comprise measuring expression of each of five to seven genes in a panel in a sample and comparing the expression of the five to seven gene with a predetermined control.

### CROSS-REFERENCE TO RELATED APPLICATION

This application claims the benefit of U.S. Provisional Application No. 63/639,782, filed Apr. 29, 2024, which is incorporated herein by reference.

### STATEMENT REGARDING FEDERALLY SPONSORED RESEARCH OR DEVELOPMENT

This invention was made with government support under Grant No. R01 GM133815 awarded by the National Institute of Health. The government has certain rights in the invention.

### BACKGROUND

Sepsis is a deadly condition without effective targeted therapies. Sepsis is a life-threatening dysregulated response to infection with no effective therapies beyond standard clinical care. Sepsis heterogeneity due to patient, pathogen, and immune-related variability, may be the causal factor for over 30 years of failed clinical trials. It has been proposed that sepsis phenotyping, to identify and target subpopulations, may increase the probability of discovering new effective therapies (Stanski N L, et al. “Prognostic and predictive enrichment in sepsis. Nature reviews Nephrology.” 2020; 16(1):20-31; Sinha P, et al. “Calfee C S. Biological Phenotyping in Sepsis and Acute Respiratory Distress Syndrome.” Annu Rev Med. 2023; 74:457-471; DeMerle K M, et al. “Sepsis Subclasses: A Framework for Development and Interpretation.” Crit Care Med. 2021; 49(5):748-759).

HDL loses its anti-inflammatory and antioxidant functions in sepsis and becomes pro-inflammatory (DysHDL). HDL becomes pro-inflammatory (DysHDL) in sepsis and DysHDL levels predict organ failure severity and death. The damaging effects of DysHDL in sepsis are due to alteration of HDL's enzymes and proteins by oxidized fatty acids and the replacement of anti-inflammatory lipoproteins with pro-inflammatory/oxidized lipids that potentiate inflammation and oxidative damage (FIG. 1). During sepsis, serum phospholipase A2 (sPLA₂) releases arachidonic acid (AA) and linoleic acid (LA) from the sn-2 position of phospholipids, which are substrates for lipoxygenase (LOX) pathway inflammatory lipids (hydroxyeicosatetraenoic, HETEs; hydroxyoctadecadienoic acids, HODEs). Studies have demonstrated the pro-inflammatory effects and lipidomic storm caused by AA metabolites and LOX products in sepsis. However, the mechanisms and potential therapeutic targets remain unknown. HETEs and HODEs are increased on HDL in the setting of inflammation, and correlate with inflammatory markers and impaired HDL function (Charles-Schoeman C, et al. “High levels of oxidized fatty acids in HDL are associated with impaired HDL function in patients with active rheumatoid arthritis. Clinical Rheumatology. 2018; 37(3):615-622). 11-HETE, 12-HETE, and 15-HETE are elevated in chronic critical illness (CCI)/early death sepsis patients.

### SUMMARY

Described are methods of treating HYPO sepsis in a subject comprising: (a) measuring expression of each of five to seven genes in a panel in a sample from the subject; (b) determining whether expression of each of the genes in the panel is increased in the sample relative to expression in a predetermined control; and (c) if expression of each of the genes in the panel is increased in the sample relative to expression in the predetermined control, then administering at least one inhibitor of at least one of the genes in the panel to the subject, wherein the panel comprises five to seven genes selected from the group consisting of: PCSK9, LDLR, PLTP, DHCR7, FDFT1, MSMO1, and ALOX5. In some embodiments, the panel of genes comprises: PCSK9, LDLR, PLTP, DHCR7, and MSMO1. In some embodiments, the panel of genes comprises: PCSK9, LDLR, PLTP, DHCR7, FDFT1, MSMO1, and ALOX5. In some embodiments, the sample comprises, consists essentially of, or consists of leukocyte cells. In some embodiments, expression of each of five to seven genes in the panel is measured in leukocyte cells.

Described are methods of determining whether a subject has HYPO sepsis or is at risk of developing HYPO sepsis. The methods comprise: measuring a level of expression of each of five to seven genes in a panel in a sample obtained from the subject, wherein the genes in the panel are selected from the group consisting of: PCSK9, LDLR, PLTP, DHCR7, FDFT1, MSMO1, and ALOX5; wherein if the level expression of each of the five to seven genes in the panel in the sample that is greater than a predetermined control then the subject is determined to have HYPO sepsis or be at risk of developing HYPO sepsis. In some embodiments, the genes in the panel comprise: PCSK9, LDLR, PLTP, DHCR7, and MSMO1. In some embodiments, the genes in the panel comprise: PCSK9, LDLR, PLTP, DHCR7, MSMO1, FDFT1, and ALOX5. In some embodiments, a subject determined to have HYPO sepsis are at risk of developing HYPO sepsis is administered at least one inhibitor of at least one of the genes in the panel. In some embodiments, the sample comprises, consists essentially of, or consists of leukocyte cells. In some embodiments, expression of each of five to seven genes in a panel is measured in leukocyte cells.

Described are methods of treating a patient with sepsis, diagnosed with sepsis, suspected of suffering sepsis, or at risk of developing sepsis comprising: (a) obtaining of having obtained a sample from the patient; (b) measuring or having measured a level of expression of each of five to seven genes in a panel in the sample, wherein the five to seven genes in the panel are selected from the group consisting of: PCSK9, LDLR, PLTP, DHCR7, FDFT1, MSMO1, and ALOX5; (c) comparing the level of expression of each of the five to seven genes in the sample with a reference level obtained from a predetermined control or standard derived from a population of healthy subjects; and (d) if the level of expression of each of the five to seven genes in a panel in the sample is greater than the reference level, then determining that the patient has HYPO sepsis or is a risk of developing HYPO sepsis and administering to the subject at least one inhibitor of at least one of the genes in the panel. In some embodiments, the genes in the panel comprise: PCSK9, LDLR, PLTP, DHCR7, and MSMO1. In some embodiments, the genes in the panel comprise: PCSK9, LDLR, PLTP, DHCR7, MSMO1, FDFT1, and ALOX5. In some embodiments, the sample comprises, consists essentially of, or consists of leukocyte cells. In some embodiments, expression of each of five to seven genes in a panel is measured in leukocyte cells.

Described are methods of treating HYPO sepsis in a subject comprising: (a) obtaining or having obtained a sample from the subject; (b) measuring or having measured a level of expression of each of five to seven genes in a panel in the sample, wherein the five to seven genes in the panel are selected from the group consisting of: PCSK9, LDLR, PLTP, DHCR7, FDFT1, MSMO1, and ALOX5; (c) determining whether the level expression of each of the five to seven genes in the panel is increased in the sample relative to a level of expression of each of the five to seven gene in the panel in a predetermined control; and (d) if the level expression of each of the five to seven genes in the panel is increased in the sample relative to the level expression of each of the five to seven genes in the predetermined control, then administering at least one inhibitor of at least one of the genes in the panel. In some embodiments, the panel of genes comprises: PCSK9, LDLR, PLTP, DHCR7, and MSMO1. In some embodiments, the panel of genes comprises: PCSK9, LDLR, PLTP, DHCR7, FDFT1, MSMO1, and ALOX5. In some embodiments, the predetermined control comprises a standard derived from a population of known healthy subjects. In some embodiments, the sample comprises, consists essentially of, or consists of leukocyte cells. In some embodiments, expression of each of five to seven genes in a panel is measured in leukocyte cells.

Inhibitors include, but are not limited to, therapeutics know in inhibit expression of a gene, decrease protein level, or decrease protein activity. Exemplary inhibitors of gene expression include, but are not limited to, antisense oligonucleotides and RNAi agents (e.g., siRNAs). Exemplary inhibitors of protein activity include, but are not limited to, substrate analogs, and antibodies.

Described are methods of identifying a subject with HYPO sepsis or at risk of developing HYPO sepsis comprising: measuring a level of expression of each of five to seven genes in a panel in a sample obtained from the subject, wherein the five to seven genes in the panel comprise: PCSK9, LDLR, PLTP, DHCR7, FDFT1, MSMO1, and ALOX5, and wherein a level of expression of each of the five to seven genes in a panel in the sample greater than a predetermined control indicates the subject has HYPO sepsis or is at risk of developing HYPO sepsis. In some embodiments, the panel of genes comprises: PCSK9, LDLR, PLTP, DHCR7, and MSMO1. In some embodiments, the panel of genes comprises: PCSK9, LDLR, PLTP, DHCR7, FDFT1, MSMO1, and ALOX5. Measuring a level of expression of each of the five to seven genes in the panel in the sample can comprise: measuring mRNA levels or measuring protein levels, or a combination thereof. In some embodiments, the predetermined control comprises a standard derived from a population of healthy subjects. In some embodiments, the sample comprises, consists essentially of, or consists of leukocyte cells. In some embodiments, expression of each of five to seven genes in a panel is measured in leukocyte cells.

In some embodiments, the methods further comprise administration of one or more additional therapies known to be effective in treating sepsis. The one or more additional therapies can be, but are not limited to: antibiotics, fluids (e.g., intravenous (I.V.) fluids), vasopressors, and insulin.

### BRIEF DESCRIPTION OF THE DRAWINGS

FIG. 1. Diagram illustration DysHDL-mediated damage to capillary endothelium in sepsis.

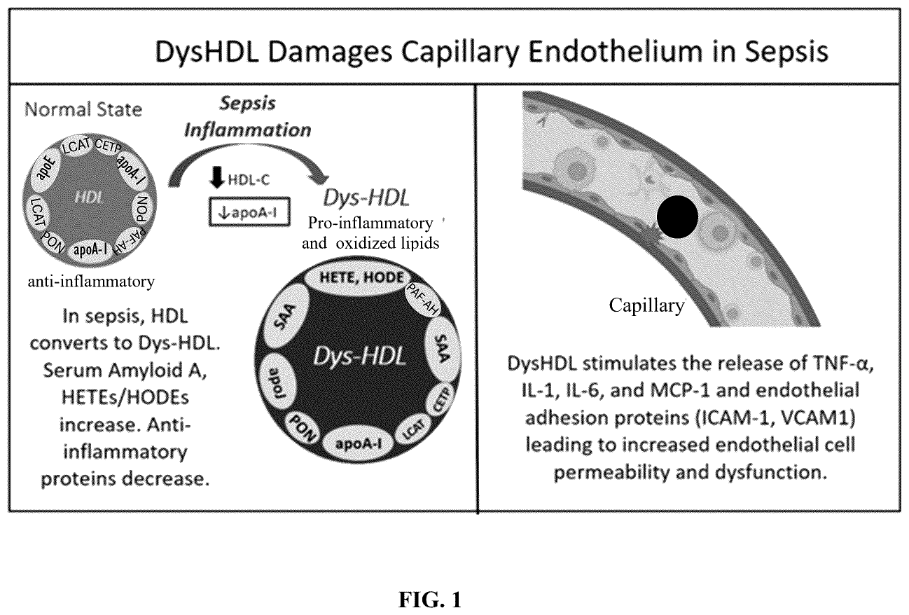

FIG. 2. Heatmap illustrating A) HYPO sepsis subjects have lower cholesterol/lipoprotein levels and higher SOFA scores and ICAM-1 levels compared to NORMO sepsis subjects (N=370). 28-day mortality: 31% HYPO, 12% NORMO patients (p<0.001). Top row indicates 28-day survival; darker=survival; lighter=non-survival. PON, paraoxonase-1; ApoA-I, apolipoprotein A-I; SOFA sequential organ failure assessment.

FIG. 3. Graph illustrating survival of HYPO sepsis subjects vs. NORMO sepsis subjects (N=192) over 1 year. HYPO sepsis subjects had significantly lower 1-year survival in comparison to NORMO sepsis subjects (p=0.0058).

FIG. 4. Graph illustrating use of a gene correlation network analysis (WGCNA) to construct a Topological Overlap Matrix (TOM) for gene co-expression analysis by subphenotype based on 5 of 7 genes: PCSK9, DHCR7, MSMO1, LDLR, and PLTP. Connections (solid lines) between DHCR7 and MSMO1 becomes strong when the status switches from NORMO to HYPO. DHCR7 is consistently connected to PCSK9 and LDLR

### DETAILED DESCRIPTION

### I. Definitions

As used herein, the singular forms “a,” “an,” and “the” include plural referents unless the content clearly dictates otherwise. Thus, for example, reference to “a polypeptide” may include two or more such molecules, and the like.

The use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls. Unless specifically noted, embodiments in the specification that recite “comprising” various components are also contemplated as “consisting of” or “consisting essentially of” the recited components.

Embodiments in the specification that recite “consisting essentially of” various components are also contemplated as “consisting of.” “Consisting essentially of” means that additional component(s), composition(s), or method step(s) that do not materially change the basic and novel characteristics of the compositions and methods described herein may be included in those compositions or methods.

As used herein, the terms “about” and “approximately,” when used to modify an amount specified in a numeric value or range, indicates the numeric value as well as reasonable deviations from the value known to the skilled person in the art (e.g., not having any significant effect on the activity or stability of the composition). In some embodiments, the term “about” means within the typical ranges of tolerances in the art. In some embodiments, the term “about” means within 1 or 2 standard deviations from the mean. In some embodiments, the term “about” means±10%. In some embodiments, the term “about” means 5%. When the term “about” is present before a series of numbers or a range, it is understood that “about” can modify each of the numbers in the series or range. When the specification discloses a specific value for a parameter, the specification should be understood as alternatively disclosing the parameter at “about” that value.

All ranges are to be interpreted as encompassing the endpoints in the absence of express exclusions, such as “not including the endpoints”; thus, for example, “within 10-15” or “from 10 to 15” includes the values 10 and 15. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In some embodiments, the subject is a human.

The terms “treat,” “treatment,” and the like, mean the methods or steps taken to provide relief from, or amelioration or alleviation of the number, severity, adverse effect, and/or frequency of one or more symptoms or pathological consequences of a disease, disorder, or condition in a subject. Treatment can be prophylactic in terms of preventing or partially preventing a disease, or a symptom or condition of the disease. Preventing includes providing prophylaxis with respect to the occurrence or recurrence of a disease in a subject that may be predisposed to the disease but has not yet been diagnosed with the disease. Preventing also includes providing prophylaxis with respect to the occurrence or recurrence of a symptom or pathological consequence of a disease in a subject that may be predisposed symptom or pathological consequence of the disease but has not yet been diagnosed with the symptom or pathological consequence the disease. Treatment can also be prophylactic in terms of delaying onset of a disease, or a symptom or condition of the disease.

Delaying development of a disease or symptom or pathological consequence of the disease indicates deferring, hindering, slowing, retarding, stabilizing, suppressing, and/or postponing development of the disease or symptom or pathological consequence of the disease. The delay can be of varying lengths of time, depending on the history of the disease and/or individual being treated. Treating can include inhibiting the disease, disorder, or condition, e.g., impeding its progress; and relieving the disease, disorder, or condition, e.g., causing regression of the disease, disorder, and/or condition. Treatment can also mean prolonging survival as compared to expected survival in the absence of treatment. Treatment can be therapeutic in terms of a partial or complete cure of a disease, condition, symptom, or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those previously diagnosed with a disease, disorder, or condition, or those identified as being at risk of developing a disease, disorder, or condition. Treating the disease, disorder, or condition can include ameliorating at least one symptom of the particular disease, disorder, or condition, even if the underlying pathophysiology is not affected.

The term “pharmaceutically acceptable excipient” refers to a non-active pharmaceutical ingredient that is biologically or pharmacologically compatible for use in humans or animals, such as but not limited to a buffer, carrier, or preservative.

An “effective amount” of an agent, or a pharmaceutical formulation containing the agent, in the context of administration, refers to an amount effective, at dosages/amounts and for periods of time necessary, to achieve a desired result, such as a therapeutic or prophylactic result.

A “therapeutically effective amount” of an agent, or a pharmaceutical formulation containing the agent, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered.

A “dose,” “unit dose,” or “dosage” refers to physically discrete units suitable for use in a subject, each unit containing a predetermined quantity of active pharmaceutical ingredient and/or a pharmaceutical composition.

The term “administer” refers to a method of delivering agents, compounds, or compositions to the desired site of biological action. These methods include, but are not limited to, topical delivery, oral delivery, parenteral delivery, intravenous delivery, intradermal delivery, intramuscular delivery, intrathecal delivery, colonic delivery, rectal delivery, or intraperitoneal delivery. In some embodiments, the compositions described herein are administered intravenously.

“Sepsis” is life-threatening organ dysfunction caused by a dysregulated host response to infection. Sepsis can be defined as SIRS in response to a known of suspected infection (pathogen). Sepsis can also be defined as an increase of 2 or more in the SOFA (Sequential Organ Failure Assessment, Table 1) or qSOFA (quick SOFA) score of a subject. Evidence of infection can be, but is not limited to, positive diagnostic test, (e.g., culture, stain, polymerase chain reaction (PCR), ELISA), a clinical syndrome pathognomonic for infection, white blood cells in a normally sterile fluid (e.g., urine or cerebrospinal fluid), evidence of a perforated viscus (free air on abdominal x-ray or CT scan; signs of acute peritonitis), abnormal chest x-ray (CXR) consistent with pneumonia (with focal opacification), petechiae, purpura, and purpura fulminans. Sepsis may be marked by the beginning of any organ dysfunction remote from a site of infection. For qSOFA, 1 point is given for each of altered mental status (Glasgow coma scale (GCS)score<15), respiratory rate≥22, and systolic blood pressure≤100 mm Hg.

TABLE 1
SOFA score

| SOFA score | 1 | 2 | 3 | 4 |
|---|---|---|---|---|
| Respiration | | | -with respiratory support- | |
| PaO₂/FiO₂ (mm Hg) | <400 | <300 | <200 | <100 |
| Coagulation | | | | |
| Platelets × 10³/mm³ | <150 | <100 | <50 | <20 |
| Liver | | | | |
| Bilirubin (mg dL⁻¹) | 1.2-1.9 | 2.0-5.9 | 6.0-11.9 | >12.0 |
| Cardiovascular | | | | |
| Hypotension | MAP* | Dopamine ≤5 or | Dopamine >5 or | Dopamine >15 or |
| | <70 | dobutamine | norepinephrine | norepinephrine |
| | | (any) | ≤0.1 | >0.1 |
| Central Nervous System | | | | |
| Glasgow Coma Score | 13-14 | 10-12 | 6-9 | <6 |
| Renal | | | | |
| Creatinine (mg dL⁻¹) or | 1.2-1.9 | 2.0-3.4 | 3.5-4.9 or <500 | >5.0 or <200 |
| urine output (mL) | | | | |

*MAP: mean arterial pressure; vasoactive mediations administered for at least 1 h (dopamine and norepinephrine μg kg⁻¹ min⁻¹).

The HYPO sepsis subphenotype represents a group of patients with a distinct clinical and biological profile based on dysregulated lipoprotein/lipid metabolism, and genetic upregulation. “HYPO sepsis” is characterized by: (a) lower lipoprotein levels or lipid dysregulation (e.g., lower HDL-C (18, IQR 64-97.3 vs. 32, IQR 20-42.5, p=<0.001), LDL-C (29, IQR 19.9-42 vs. 48.5, IQR 34.1-71, p=<0.001), total cholesterol (78, IQR 64-97.3 vs. 107, IQR 87-133.8, p=<0.001), ApoA-I, Apo-B, and PON levels compared to levels in NORMO sepsis subjects), (b) increased endothelial dysfunction (increased ICAM-1 levels compared to levels in NORMO sepsis subjects), (c) increased vasopressor use (67% vs 34%, p=<0.001), and (d) higher SOFA scores compared to NORMO sepsis subjects (9, IQR 7-11 vs 5, IQR 4-7.5, p=<0.001). HYPO sepsis subjects may also have significantly lower levels of cholesterol esters (CE, q-value=0.000), lysophosphatidylcholines (LPC, q=0.000; LPC16:0, LPC18:1, LPC18:2), and/or sphingomyelins (SM, q=0.0005), and decreased neutrophil bactericidal activity. A hypolipoprotein (HYPO) sepsis phenotype indicates reduced lipoprotein antioxidant capacity, increased endothelial dysfunction and organ failure, a greater risk for organ failure or chronic critical illness (CCI), and worse clinical outcomes (e.g., early death (higher 28-day mortality: 30% vs 16%, p=0.004)).

### II. Overview

Sepsis occurs when a subject's immune system has a dangerous reaction to an infection. Extensive inflammation throughout the body can lead to tissue damage, organ failure, and even death. Sepsis happens when an existing infection triggers a chain reaction throughout the body. Infections that lead to sepsis most often start in the lung, urinary tract, skin, or gastrointestinal tract. Without timely treatment, sepsis can rapidly lead to tissue damage, organ failure, and death.

Described is a five to seven gene panel for use in diagnosis of HYPO sepsis or risk of HYPO sepsis, wherein the genes of the five to seven gene panel are selected from the group consisting of: the PCSK9 gene, the LDLR gene, the PLTP gene, the DHCR7 gene, the FDFT1 gene, the MSMO1 gene, and the ALOX5 gene. In some embodiments, the panel of genes comprises: PCSK9, LDLR, PLTP, DHCR7, and MSMO1. In some embodiments, the panel of genes comprises: PCSK9, LDLR, PLTP, DHCR7, FDFT1, MSMO1, and ALOX5. The genes of the five to seven gene panel play roles in endotoxin clearance, cholesterol biosynthesis, and immune regulation that are upregulated in HYPO sepsis patients.

In some embodiments, the level of expression of the genes in the five to seven gene panel in a subject is compared with a predetermined control. A level of expression of the genes in the five to seven gene panel as measured in the sample obtained from the subject that is higher relative to the level of expression of the genes in the five to seven gene panel in the predetermined control indicates the subject has HYPO sepsis or is at risk of developing HYPO sepsis. In some embodiments, a level of expression of each of the genes in the five to seven gene panel as measured in the sample obtained from the subject that is higher relative to the level of expression of the corresponding gene in the five to seven gene panel in the predetermined control indicates the subject has HYPO sepsis or is at risk of developing HYPO sepsis (e.g., expression of PCSK9 in the sample is higher relative to expression of PCSK9 in the predetermined control, expression of LDLR in the sample is higher relative to expression of LDLR in the predetermined control, expression of PLTP in the sample is higher relative to expression of in the predetermined control, expression of DHCR7 in the sample is higher relative to expression of in the predetermined control, expression of MSMO1 in the sample is higher relative to expression of MSMO1 in the predetermined control, expression of FDFT1 in the sample is higher relative to expression of FDFT1 in the predetermined control, and expression of ALOX5 in the sample is higher relative to expression of ALOX5 in the predetermined control). In some embodiments, the five to seven genes are upregulated in circulating leukocytes.

Described are compounds and methods for identifying and treating HYPO sepsis. Treating HYPO sepsis can include, but is not limited to, ameliorating one or more symptoms associated with HYPO sepsis, reducing the severity of HYPO sepsis, reducing the duration of HYPO sepsis, preventing HYPO sepsis, and/or preventing the progression of HYPO sepsis to chronic critical illness (CCI) or early death. In some embodiments, treating HYPO sepsis comprises: reducing the risk of mortality associated with or caused by HYPO sepsis, increasing survival rate of a subject with or at risk of developing HYPO sepsis, decreasing the morbidity and mortality caused by HYPO sepsis, preventing progression from HYPO sepsis to severe sepsis or septic shock, or preventing or decreasing the risk of multiple organ dysfunction syndrome associated with HYPO sepsis.

In some embodiments, the methods comprise identifying a subject having HYPO sepsis and administering to the subject one or more compounds that inhibits one or more of: PCSK9, LDLR, PLTP, DHCR7, FDFT1, MSMO1, and ALOX5. Inhibiting one or more of PCSK9, LDLR, PLTP, DHCR7, FDFT1, MSMO1, and ALOX includes (a) inhibiting expression of one or more of a PCSK9 gene, a LDLR gene, a PLTP gene, a DHCR7 gene, a FDFT1 gene, a MSMO1 gene, and a ALOX gene; or (b) inhibiting activity of one or more of PCSK9, LDLR, PLTP, DHCR7, FDFT1, MSMO1, and ALOX (proteins encoded by the PCSK9 gene, the LDLR gene, the PLTP gene, the DHCR7 gene, the FDFT1 gene, the MSMO1 gene, and the ALOX gene, respectively). Identifying a subject having HYPO sepsis comprises measuring expression of each of the PCSK9 gene, the LDLR gene, the PLTP gene, the DHCR7 gene, and the MSMO1 gene in a sample from the subject (five gene panel); and determining whether expression of each of the genes in the five gene panel is increased in the sample relative to expression in a predetermined control, wherein if expression of each of the genes in the five gene panel is increased in the sample relative to expression in the predetermined control, then determining the subject has HYPO sepsis. In some embodiments, a identifying a subject having HYPO sepsis further comprises measuring expression of each of the FDFT1 gene and the ALOX5 gene in a sample from the subject (seven gene panel); and determining whether expression of each of the genes in the seven gene panel is increased in the sample relative to expression in a predetermined control, wherein if expression of each of the genes in the seven gene panel is increased in the sample relative to expression in the predetermined control, then determining the subject has HYPO sepsis. In some embodiments, the sample comprises, consists essentially of, or consists of leukocyte cells. In some embodiments, expression of each of genes in the five to seven gene panel is measured in leukocyte cells.

In some embodiments, the methods comprise identifying a subject having sepsis or suspected of having sepsis as being at risk of HYPO sepsis and administering to the subject one or more compounds that inhibits one or more of: PCSK9, LDLR, PLTP, DHCR7, FDFT1, MSMO1, and ALOX5. Inhibiting one or more of PCSK9, LDLR, PLTP, DHCR7, FDFT1, MSMO1, and ALOX includes (a) inhibiting expression of one or more of a PCSK9 gene, a LDLR gene, a PLTP gene, a DHCR7 gene, a FDFT1 gene, a MSMO1 gene, and a ALOXgene; or (b) inhibiting activity of one or more of PCSK9, LDLR, PLTP, DHCR7, FDFT1, MSMO1, and ALOX (proteins encoded by the PCSK9 gene, the LDLR gene, the PLTP gene, the DHCR7 gene, the FDFT1 gene, the MSMO1 gene, and the ALOX gene, respectively). In some embodiments, identifying a subject having sepsis or suspected of having sepsis as being at risk of HYPO sepsis comprises measuring expression of each of the PCSK9 gene, the LDLR gene, the PLTP gene, the DHCR7 gene, and the MSMO1 gene (five gene panel) in a sample from the subject; and determining whether expression of each of the genes in the five gene panel is increased in the sample relative to expression in a predetermined control, wherein if expression of each of the genes in the five gene panel is increased in the sample relative to expression in the predetermined control, then determining the subject has HYPO sepsis. In some embodiments, identifying a subject having sepsis or suspected of having sepsis as being at risk of HYPO sepsis further comprises measuring expression of each of the FDFT1 gene and the ALOX5 gene (seven gene panel) in a sample from the subject; and determining whether expression of each of the genes in the seven gene panel is increased in the sample relative to expression in a predetermined control, wherein if expression of each of the genes in the seven gene panel is increased in the sample relative to expression in the predetermined control, then determining the subject has HYPO sepsis. In some embodiments, the sample comprises, consists essentially of, or consists of leukocyte cells. In some embodiments, expression of each of genes in the five to seven gene panel is measured in leukocyte cells.

In some embodiments, the methods comprise identifying a subject at risk of developing HYPO sepsis and administering to the subject one or more compounds that inhibits one or more of: PCSK9, LDLR, PLTP, DHCR7, FDFT1, MSMO1, and ALOX5. Inhibiting one or more of PCSK9, LDLR, PLTP, DHCR7, FDFT1, MSMO1, and ALOX includes (a) inhibiting expression of one or more of a PCSK9 gene, a LDLR gene, a PLTP gene, a DHCR7 gene, a FDFT1 gene, a MSMO1 gene, and a ALOXgene; or (b) inhibiting activity of one or more of PCSK9, LDLR, PLTP, DHCR7, FDFT1, MSMO1, and ALOX (proteins encoded by the PCSK9 gene, the LDLR gene, the PLTP gene, the DHCR7 gene, the FDFT1 gene, the MSMO1 gene, and the ALOX gene, respectively). In some embodiments, identifying a subject at risk of developing HYPO sepsis comprises measuring expression of each of the PCSK9 gene, the LDLR gene, the PLTP gene, the DHCR7 gene, and the MSMO1 gene (five gene panel) in a sample from the subject; and determining whether expression of each of the genes in the five gene panel is increased in the sample relative to expression in a predetermined control, wherein if expression of each of the genes in the five gene panel is increased in the sample relative to expression in the predetermined control, then determining the subject has HYPO sepsis. In some embodiments, identifying a subject at risk of developing HYPO sepsis further comprises measuring expression of each of the FDFT1 gene and the ALOX5 gene (seven gene panel) in a sample from the subject; and determining whether expression of each of the genes in the seven gene panel is increased in the sample relative to expression in a predetermined control, wherein if expression of each of the genes in the seven gene panel is increased in the sample relative to expression in the predetermined control, then determining the subject has HYPO sepsis. In some embodiments, the sample comprises, consists essentially of, or consists of leukocyte cells. In some embodiments, expression of each of genes in the five to seven gene panel is measured in leukocyte cells.

In some embodiments, the methods for treating a subject suffering from or suspected of suffering sepsis comprise: (a) measuring expression of each gene in a five to seven gene panel in a sample from the subject; (b) determining whether expression of each of the genes in the five to seven gene panel is increased in the sample relative to expression of the same gene in a predetermined control; and (c) if expression of each of the genes in the five to seven gene panel is increased in the sample relative to expression in the predetermined control, then administering at least one inhibitor of at least one of the genes in the five to seven gene panel to the subject, wherein the five to seven genes in the panel are selected from the group consisting of: a PCSK9 gene, a LDLR gene, a PLTP gene, a DHCR7 gene, a FDFT1 gene, a MSMO1 gene, and a ALOX5 gene. In some embodiments, the panel of genes comprises: PCSK9, LDLR, PLTP, DHCR7, and MSMO1. In some embodiments, the panel of genes comprises: PCSK9, LDLR, PLTP, DHCR7, FDFT1, MSMO1, and ALOX5. Inhibiting one or more of PCSK9, LDLR, PLTP, DHCR7, FDFT1, MSMO1, and ALOX includes (a) inhibiting expression of one or more of a PCSK9 gene, a LDLR gene, a PLTP gene, a DHCR7 gene, a FDFT1 gene, a MSMO1 gene, and a ALOX gene; or (b) inhibiting activity of one or more of PCSK9, LDLR, PLTP, DHCR7, FDFT1, MSMO1, and ALOX (proteins encoded by the PCSK9 gene, the LDLR gene, the PLTP gene, the DHCR7 gene, the FDFT1 gene, the MSMO1 gene, and the ALOX gene, respectively). In some embodiments, the sample comprises, consists essentially of, or consists of leukocyte cells. In some embodiments, expression of each of five to seven genes in a panel is measured in leukocyte cells.

In some embodiments, the methods for treating a subject suffering from or suspected of suffering HYPO sepsis comprise: (a) measuring expression of each gene in a five to seven gene panel in a sample from the subject; (b) determining whether expression of each of the genes in the five to seven gene panel is increased in the sample relative to expression in a predetermined control; and (c) if expression of each of the genes in the five to seven gene panel is increased in the sample relative to expression in the predetermined control, then determining the subject has HYPO sepsis and administering to the subject at least one inhibitor of at least one of the genes in the five to seven gene panel, wherein the five to seven genes are selected from the group consisting of: a PCSK9 gene, a LDLR gene, a PLTP gene, a DHCR7 gene, a FDFT1 gene, a MSMO1 gene, and a ALOX5 gene. In some embodiments, the panel of genes comprises: PCSK9, LDLR, PLTP, DHCR7, and MSMO1. In some embodiments, the panel of genes comprises: PCSK9, LDLR, PLTP, DHCR7, FDFT1, MSMO1, and ALOX5. Inhibiting one or more of PCSK9, LDLR, PLTP, DHCR7, FDFT1, MSMO1, and ALOX includes (a) inhibiting expression of one or more of a PCSK9 gene, a LDLR gene, a PLTP gene, a DHCR7 gene, a FDFT1 gene, a MSMO1 gene, and a ALOX gene; or (b) inhibiting activity of one or more of PCSK9, LDLR, PLTP, DHCR7, FDFT1, MSMO1, and ALOX (proteins encoded by the PCSK9 gene, the LDLR gene, the PLTP gene, the DHCR7 gene, the FDFT1 gene, the MSMO1 gene, and the ALOX gene, respectively). In some embodiments, the sample comprises, consists essentially of, or consists of leukocyte cells. In some embodiments, expression of each of five to seven genes in a panel is measured in leukocyte cells.

In some embodiments, the methods for treating a subject at risk of developing HYPO sepsis comprise: (a) measuring expression of each gene in a five to seven gene panel in a sample from the subject; (b) determining whether expression of each of the genes in the five to seven gene panel is increased in the sample relative to expression in a predetermined control; and (c) if expression of each of the genes in the five to seven gene panel is increased in the sample relative to expression in the predetermined control, then determining the subject is at risk of developing HYPO sepsis and administering to the subject at least one inhibitor of at least one of the genes in the five to seven gene panel, wherein the five to seven genes are selected from the group consisting of: a PCSK9 gene, a LDLR gene, a PLTP gene, a DHCR7 gene, a FDFT1 gene, a MSMO1 gene, and a ALOX5 gene. In some embodiments, the panel of genes comprises: PCSK9, LDLR, PLTP, DHCR7, and MSMO1. In some embodiments, the panel of genes comprises: PCSK9, LDLR, PLTP, DHCR7, FDFT1, MSMO1, and ALOX5. Inhibiting one or more of PCSK9, LDLR, PLTP, DHCR7, FDFT1, MSMO1, and ALOX includes (a) inhibiting expression of one or more of a PCSK9 gene, a LDLR gene, a PLTP gene, a DHCR7 gene, a FDFT1 gene, a MSMO1 gene, and a ALOX gene; or (b) inhibiting activity of one or more of PCSK9, LDLR, PLTP, DHCR7, FDFT1, MSMO1, and ALOX (proteins encoded by the PCSK9 gene, the LDLR gene, the PLTP gene, the DHCR7 gene, the FDFT1 gene, the MSMO1 gene, and the ALOX gene, respectively). In some embodiments, the sample comprises, consists essentially of, or consists of leukocyte cells. In some embodiments, expression of each of five to seven genes in a panel is measured in leukocyte cells.

In some embodiments, the methods for preventing HYPO sepsis in a subject comprise: (a) measuring expression of each gene in a five to seven gene panel in a sample from the subject; (b) determining whether expression of each of the genes in the five to seven gene panel is increased in the sample relative to expression in a predetermined control; and (c) if expression of each of the genes in the five to seven gene panel is increased in the sample relative to expression in the predetermined control, then determining the subject is at risk of developing HYPO sepsis and administering to the subject at least one inhibitor of at least one of the genes in the five to seven gene panel, wherein the five to seven genes are selected from the group consisting of: a PCSK9 gene, a LDLR gene, a PLTP gene, a DHCR7 gene, a FDFT1 gene, a MSMO1 gene, and a ALOX5 gene. In some embodiments, the panel of genes comprises: PCSK9, LDLR, PLTP, DHCR7, and MSMO1. In some embodiments, the panel of genes comprises: PCSK9, LDLR, PLTP, DHCR7, FDFT1, MSMO1, and ALOX5. Inhibiting one or more of PCSK9, LDLR, PLTP, DHCR7, FDFT1, MSMO1, and ALOX includes (a) inhibiting expression of one or more of a PCSK9 gene, a LDLR gene, a PLTP gene, a DHCR7 gene, a FDFT1 gene, a MSMO1 gene, and a ALOX gene; or (b) inhibiting activity of one or more of PCSK9, LDLR, PLTP, DHCR7, FDFT1, MSMO1, and ALOX (proteins encoded by the PCSK9 gene, the LDLR gene, the PLTP gene, the DHCR7 gene, the FDFT1 gene, the MSMO1 gene, and the ALOX gene, respectively). In some embodiments, the sample comprises, consists essentially of, or consists of leukocyte cells. In some embodiments, expression of each of five to seven genes in a panel is measured in leukocyte cells.

In some embodiments, the methods for reducing the risk of mortality associated with HYPO sepsis in a subject or increasing survival rate of a subject with or at risk of developing HYPO sepsis comprise: (a) measuring expression of each gene in a five to seven gene panel in a sample from the subject; (b) determining whether expression of each of the genes in the five to seven gene panel is increased in the sample relative to expression in a predetermined control; and (c) if expression of each of the genes in the five to seven gene panel is increased in the sample relative to expression in the predetermined control, then administering to the subject at least one inhibitor of at least one of the genes in the five to seven gene panel, wherein the five to seven genes are selected from the group consisting of: a PCSK9 gene, a LDLR gene, a PLTP gene, a DHCR7 gene, a FDFT1 gene, a MSMO1 gene, and a ALOX5 gene. In some embodiments, the panel of genes comprises: PCSK9, LDLR, PLTP, DHCR7, and MSMO1. In some embodiments, the panel of genes comprises: PCSK9, LDLR, PLTP, DHCR7, FDFT1, MSMO1, and ALOX5. Inhibiting one or more of PCSK9, LDLR, PLTP, DHCR7, FDFT1, MSMO1, and ALOX includes (a) inhibiting expression of one or more of a PCSK9 gene, a LDLR gene, a PLTP gene, a DHCR7 gene, a FDFT1 gene, a MSMO1 gene, and a ALOX gene; or (b) inhibiting activity of one or more of PCSK9, LDLR, PLTP, DHCR7, FDFT1, MSMO1, and ALOX (proteins encoded by the PCSK9 gene, the LDLR gene, the PLTP gene, the DHCR7 gene, the FDFT1 gene, the MSMO1 gene, and the ALOX gene, respectively). In some embodiments, the sample comprises, consists essentially of, or consists of leukocyte cells. In some embodiments, expression of each of five to seven genes in a panel is measured in leukocyte cells.

In some embodiments, the methods for preventing progression from HYPO sepsis to severe sepsis or septic shock or preventing or decreasing the risk of multiple organ dysfunction syndrome associated with HYPO sepsis in a subject comprise: (a) measuring expression of each gene in a five to seven gene panel in a sample from the subject; (b) determining whether expression of each of the genes in the five to seven gene panel is increased in the sample relative to expression in a predetermined control; and (c) if expression of each of the genes in the five to seven gene panel is increased in the sample relative to expression in the predetermined control, then administering to the subject at least one inhibitor of at least one of the genes in the five to seven gene panel, wherein the five to seven genes are selected from the group consisting of: a PCSK9 gene, a LDLR gene, a PLTP gene, a DHCR7 gene, a FDFT1 gene, a MSMO1 gene, and a ALOX5 gene. In some embodiments, the panel of genes comprises: PCSK9, LDLR, PLTP, DHCR7, and MSMO1. In some embodiments, the panel of genes comprises: PCSK9, LDLR, PLTP, DHCR7, FDFT1, MSMO1, and ALOX5. Inhibiting one or more of PCSK9, LDLR, PLTP, DHCR7, FDFT1, MSMO1, and ALOX includes (a) inhibiting expression of one or more of a PCSK9 gene, a LDLR gene, a PLTP gene, a DHCR7 gene, a FDFT1 gene, a MSMO1 gene, and a ALOX gene; or (b) inhibiting activity of one or more of PCSK9, LDLR, PLTP, DHCR7, FDFT1, MSMO1, and ALOX (proteins encoded by the PCSK9 gene, the LDLR gene, the PLTP gene, the DHCR7 gene, the FDFT1 gene, the MSMO1 gene, and the ALOX gene, respectively). In some embodiments, the sample comprises, consists essentially of, or consists of leukocyte cells. In some embodiments, expression of each of five to seven genes in a panel is measured in leukocyte cells.

In some embodiments, the sample is obtained from a subject suffering from, or diagnosed with, sepsis. In some embodiments, the sample is obtained from a subject suffering from, or diagnoses with, at least one symptom of sepsis. In some embodiments, the sample is obtained from a subject suspected of suffering from sepsis. In some embodiments, the sample is obtained from a subject suspected of suffering from at least one symptom of sepsis. In some embodiments, the sample is obtained from a subject at risk of developing sepsis. In some embodiments, the sample is obtained from a subject at risk of developing at least one symptom of sepsis. In some embodiments, the sample comprises, consists essentially of, or consists of leukocyte cells. In some embodiments, expression of each of five to seven genes in a panel is measured in leukocyte cells.

Measuring expression of the gene in the five to seven gene panel can be determined by measuring mRNA (measuring gene expression) in the sample, measuring protein in the sample, measuring protein activity in the sample, or a combination thereof. Measuring expression can be assessed by any method (e.g., test or assay) known in the art for measuring gene expression levels or protein levels. Exemplary methods of measuring gene expression levels include, but are not limited to, nucleic acid amplification assays, polymerase chain reaction (PCR) assays, real time PCR, TaqMan-based assays, qPCR, qRT-PCR, microarray analysis, LAMP assay, hybridization assays (e.g., Northern blotting), and RNA-Seq. Exemplary methods of measuring protein levels include, but are not limited to, immune-based detection assays (e.g., Enzyme-Linked Immunosorbent Assays (ELISA), AlphaLISA, ELISPOT, Western blotting, or FISH), enzymatic activity measurement (protein activity assay). In some embodiments, the test or assay is an FDA-approved test or assay.

The level of expression of the genes in the five to seven gene panel as measured or determined in the sample from the subject is compared with the level of expression of the genes in the five to seven gene panel in a predetermined control. In some embodiments, the predetermined control comprises a standard derived from a population of known healthy subjects. The level of expression of the genes in the five to seven gene panel as determined for the population of healthy subjects is measured using the same or substantially the same method as used to measure the level of the genes in the five to seven gene panel in the sample obtained from the subject.

The inhibitor can be a compound (i.e., molecule or therapeutic agent) known or reported to inhibit activity of PCSK9, LDLR, PLTP, DHCR7, FDFT1, MSMO1, or ALOX, or to inhibit expression of PCSK9, LDLR, PLTP, DHCR7, FDFT1, MSMO1, or ALOX. The inhibitor can also be a compound known to block a product or action of a product of PCSK9, LDLR, PLTP, DHCR7, FDFT1, MSMO1, or ALOX. In some embodiments, the inhibitor comprises a compound (i.e., active pharmaceutical ingredient) approved by a regulatory agency that acts by inhibiting activity of PCSK9, LDLR, PLTP, DHCR7, FDFT1, MSMO1, or ALOX or by inhibiting expression of PCSK9, LDLR, PLTP, DHCR7, FDFT1, MSMO1, or ALOX.

### III. Five to Seven Gene Panel and Known Inhibitors

The genes in the five to seven gene panel are selected from the group consisting of: the PCSK9 gene, the LDLR gene, the PLTP gene, the DHCR7 gene, the FDFT1 gene, the MSMO1 gene, and the ALOX5 gene. In some embodiments, the panel of genes comprises: PCSK9, LDLR, PLTP, DHCR7, and MSMO1. In some embodiments, the panel of genes comprises: PCSK9, LDLR, PLTP, DHCR7, FDFT1, MSMO1, and ALOX5.

1. PCSK9: The PCSK9 gene encodes proprotein convertase subtilisin/kexin type 9 (PCSK9), a member of the subtilisin-like proprotein convertase family, which includes proteases that process protein and peptide precursors trafficking through regulated or constitutive branches of the secretory pathway. PCSK9 enables endotoxin clearance via the LDL receptor and promotes endothelial dysfunction via TLR4/MyD88/NF-κB and NLRP3 pathways.

Drugs known to inhibit PCSK9 include, but are not limited to, evolocumab, alirocumab, bococizumab, 1D05-IgG2 (Merck), RG-7652, LY3015014, and inclisiran.

Evolocumab, alirocumab, bococizumab are monoclonal antibodies. Alirocumab is FDA approved.

Inclisiran is an RNAi therapeutic.

Additional PCSK9 inhibitors are described in: Evison B J et al. “A small molecule inhibitor of PCSK9 that antagonizes LDL receptor binding via interaction with a cryptic PCSK9 binding groove” Bioorg Med Chem. 2020 Mar. 15; 28(6):115344. Epub 2020 Jan. 31; and Suchowerska A K et al. “A Novel, Orally Bioavailable, Small-Molecule Inhibitor of PCSK9 With Significant Cholesterol-Lowering Properties In Vivo.” J Lipid Res. 2022 November; 63(11):100293. Epub 2022 Oct. 6.

2. LDLR: The LDLR gene encodes the LDL receptor (LDLR), which mediates endocytosis of cholesterol-rich low-density lipoprotein (LDL).

3. PLTP: The PLTP gene encodes plasma phospholipid transfer protein (PLTP), which transfers phospholipids from triglyceride-rich lipoproteins (VLDL, LDL) to HDL, regulating HDL's size. PLTP plays a role in endotoxin clearance and immune modulation.

PLTP inhibitors are described in: Luo Y et al. “Pharmacologic Inhibition of Phospholipid Transfer Protein Activity Reduces Apolipoprotein-B Secretion from Hepatocytes.” J Pharmacol Exp Ther. 2010 March; 332(3):1100-1106; and Hantani R et al. “Identification of Novel Phospholipid Transfer Protein Inhibitors by High-Throughput Screening” SLAS Discovery June 2019, 24(5):579-586.

4. DHCR7: The DHCR7 gene encodes 7-dehydrocholesterol reductase (DHCR7), which catalyzes a critical step in cholesterol biosynthesis and is a precursor for vitamin D.

Drugs that inhibit DHCR7 include, but are not limited to, AY9944, ziprasidone, ketanserin, pergolide, vilazodone, brexpiprazole, indacaterol, cholic acid, and tamoxifen.

In some embodiments, the DHCR7 inhibitor comprises AY9944 [trans-1,4-bis(2-chloro-benzylaminomethyl) (CAS No. 366-93-8) (Wang H et al. “Targeting 7-dehydrocholesterol reductase against EV-A71 replication by upregulating interferon response” Antiviral Res 2023 January:209:105497.

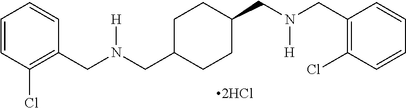

In some embodiments, the DHCR7 inhibitor comprises an atypical antipsychotic. Atypical antipsychotics, also known as second generation antipsychotics (SGAs) or serotonin-dopamine antagonists (SDAs), block receptors in the brain's dopamine pathways. Atypical antipsychotics are an antipsychotic drug typically used to treat psychiatric conditions. Atypical antipsychotics include, but are not limited to, ziprasidone, brexpiprazole, clozapine, cariprazine, perospirone, nefazodone, aripiprazole, and risperidone. In some embodiments, the Dhcr7 inhibitor comprises ziprasidone. In some embodiments, the Dhcr7 inhibitor comprises brexpiprazole.

(a) ziprasidone (CAS No. 146939-27-7) (Ghersi D et al. “Identifying Molecular Fragments That Drive 7-Dehydrocholesterol Elevation” ACS Pharmacol Transl Sci. 2022 Jan. 14; 5(1): 3-7)

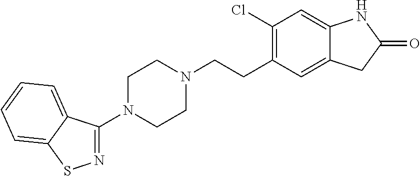 [SMILES: O=C1Cc2cc(CCN3CCN(c4nsc5ccccc45)CC3)c(Cl)cc2N1]

b) brexpiprazole (CAS No. 913611-97-9) (Genaro-Mattos T C et al. “Dichlorophenyl piperazines, including a recently approved atypical antipsychotic, are potent inhibitors of DHCR7, the last enzyme in cholesterol biosynthesis” Toxicol Appl Pharmacol. 2018 Jun. 15; 349: 21-28).

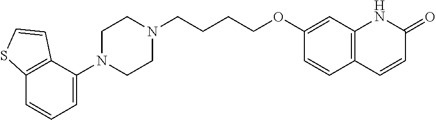 [SMILES: O=c1ccc2ccc(OCCCCN3CCN(c4cccc5sccc45)CC3)cc2[nH]1]

In some embodiments, the DHCR7 inhibitor comprises a 5-HT_{2A} receptor antagonist. 5-HT_{2A} receptor antagonists include, but are not limited to, ketanserin. In some embodiments, the Dhcr7 inhibitor comprises ketanserin.

(a) ketanserin (CAS. No. 74050-98-9)

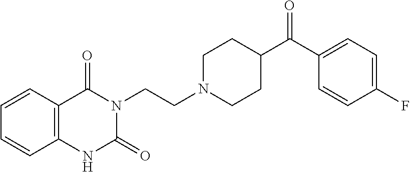 [SMILES: O=C(c1ccc(F)cc1)C1CCN(CCn2c(=O)[nH]c3ccccc3c2=O)CC1]

In some embodiments, the DHCR7 inhibitor comprises a dopamine receptor agonist. Dopamine receptor agonists include, but are not limited to, pergolide. In some embodiments, the Dhcr7 inhibitor comprises pergolide.

(a) pergolide (CAS. No. 66104-22-1) (Wages P A et al. “Identification and characterization of prescription drugs that change levels of 7-dehydrocholesterol and desmosterol” J Lipid Res. 2018 October; 59(10): 1916-1926.)

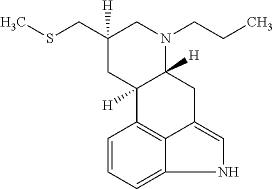 [SMILES: CCCN1C[C@H](CSC)C[C@@H]2c3cccc4[nH]cc(c34)C[C@H]21]

In some embodiments, the DHCR7 inhibitor comprises a β-2 adrenergic receptor agonist. β-2 adrenergic receptor agonists include, but are not limited to, indacaterol and metoprolol. In some embodiments, the Dhcr7 inhibitor comprises indacaterol.

(a) indacaterol (CAS. No. 312753-06-3)

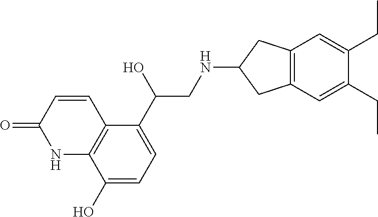 [SMILES: CCc1cc2c(cc1CC)CC(NCC(O)c1ccc(O)c3[nH]c(=O)ccc13)C2]

In some embodiments, the DHCR7 inhibitor comprises a selective serotonin reuptake inhibitor (SSRI). SSRIs include, but are not limited to, vilazodone, fluoxetine, and trazodone. In some embodiments, the Dhcr7 inhibitor comprises vilazodone.

(a) vilazodone (CAS. No. 163521-12-8) (Ghersi D et al. 2022)

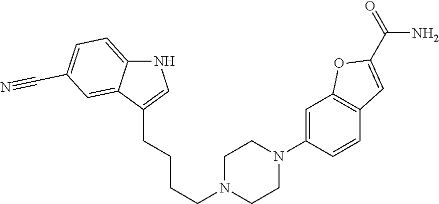 [SMILES: N#Cc1ccc2[nH]cc(CCCCN3CCN(c4ccc5cc(C(N)=O)oc5c4)CC3)c2c1]

Additional compounds (i.e., drugs) known to inhibit Dhcr7 include, but are not limited to, tamoxifen, haloperidol, ifenprodil, cyclopamine, doxorubicin, BM 15.766, and GANT61. In some embodiments, the Dhcr7 inhibitor comprises tamoxifen.

(a) tamoxifen (CAS. No. 10540-29-1) (Xiao J et al. “Targeting 7-Dehydrocholesterol Reductase Integrates Cholesterol Metabolism and IRF3 Activation to Eliminate Infection” Immunity. 2020 Jan. 14; 52(1):109-122.e6; Boland M R et al. 2016)

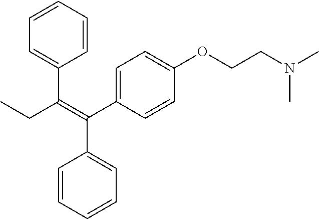 [SMILES: CC/C(=C(\c1ccccc1)c1ccc(OCCN(C)C)cc1)c1ccccc1]

Additional FDA-approved drugs that may inhibit DHCR7 include, but are not limited to, Fluphenazine, Letrozole, Sildenafil, Melperone, Zopiclone, Chlorcyclizine, Vardenafil, Donepezil, Fentanyl, Lurasidone, Piperaquine, Itraconazole, Flunarizine, Lumateperone, Prazosin, Piperazine, Asenapine, Quetiapine, Cinnarizine, Azatadine, Olanzapine, Meclizine, Cyproheptadine, Cetirizine, Vandetanib, Promazine, Gilteritinib, Lomitapide, Zuclopenthixol, Promethazine, Paliperidone, Oxybutynin, and Loperamide.

5. MSMO1: The MSMO1 gene encodes methylsterol monooxygenase 1 (MSMO1), which catalyzes three-step mono-oxygenation required for the demethylation of 4,4-dimethyl and 4-alpha-methylsterols, which can be subsequently metabolized to cholesterol.

6. FDFT1: The FDFT1 gene encodes farnesyl-diphosphate farnesyltransferase 1 (FDFT1), a membrane-associated enzyme located at a branch point in the mevalonate pathway, the first enzyme in cholesterol biosynthesis, catalyzing formation of squalene.

Inhibitors of FDFT1 include, but are not limited to, structural analogues of farnesyl pyrophosphate (e.g., ER-28488 and ER-27856), quinuclidine derivatives (e.g., RPR 107393 and YM-53601), schizostatin, zaragozic acid A, benzoxazepines (e.g., TAK-475), squalene synthase inhibitors (e.g., DF-461), and bavachinin.

Additional FDFT1 inhibitors are described in: Ha N T et al. “Roles of Farnesyl-Diphosphate Farnesyltransferase 1 in Tumour and Tumour Microenvironments” Cells. 2020 November; 9(11):2352. Published online 2020 Oct. 25.

7. ALOX5: The ALOX5 gene encodes arachidonate 5-lipoxygenase (ALOX5), which generates 5-HETE, which leads to leukotriene production. Knockout of 5LOX (encoded enzyme) has been shown to protect against myocardial injury, sepsis-mediated inflammation, oxidative stress, and apoptosis (Xie S, et al. “Inhibition of 5-lipoxygenase is associated with downregulation of the leukotriene B4 receptor 1/Interleukin-12p35 pathway and ameliorates sepsis-induced myocardial injury. Free Radic Biol Med. 2021; 166:348-357).

Inhibitors of ALOX5 (5-lipooxygenase inhibitors) include, but are not limited to, zileuton, montelukast, meclofenamate sodium, and the natural products myxochelins/pseudochelin and nordihydroguaiaretic acid (NDGA). Zileutin is FDA approved for the treatment of asthma.

### EXAMPLES

### Example 1. HYPO Sepsis Phenotype

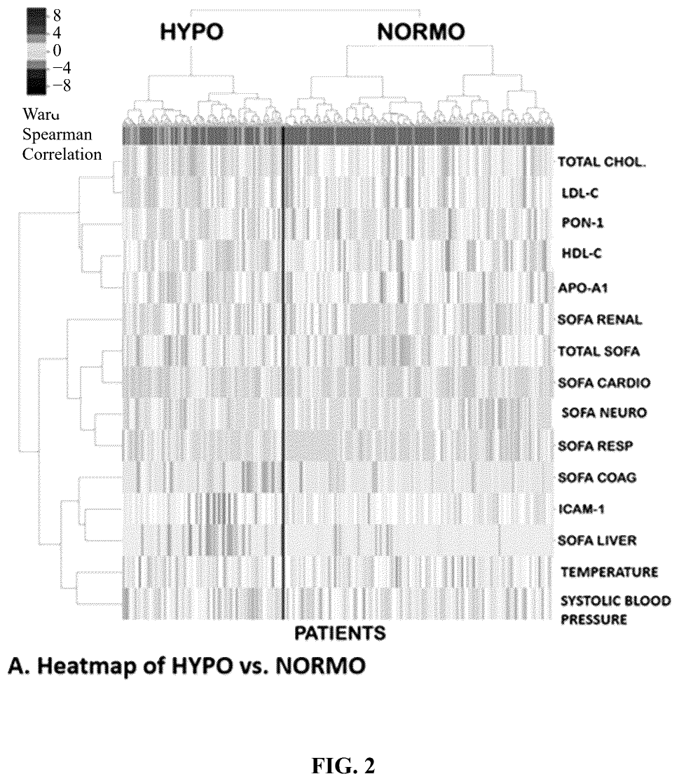

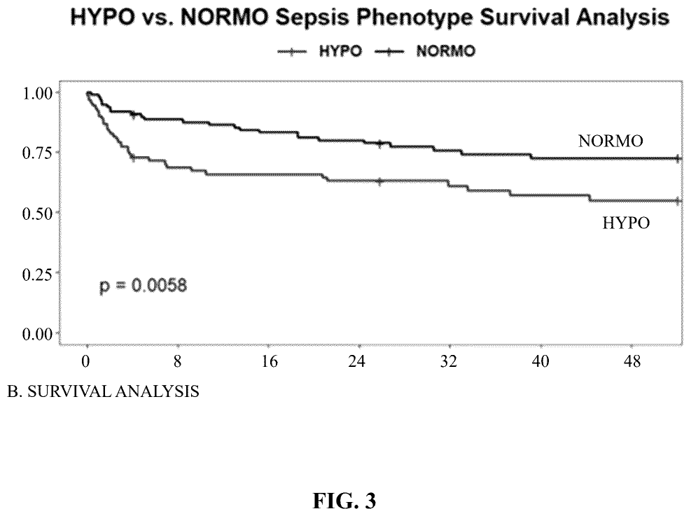

A machine learning (ML) pipeline was applied to phenotype new sepsis patients. An initial HYPO sepsis subphenotype study included 258 sepsis patients from which we derived our ML pipeline using 15 clinical features, lipoproteins/cholesterol, and biomarkers (FIG. 2). Subsequently 112 additional sepsis patients were phenotyped (total N=370, 138 HYPO sepsis, 232 NORMO sepsis). Analysis of 192 patients with one year survival data showed HYPO sepsis patients had significantly lower one year survival compared to NORMO sepsis patients (p=0.0058) (FIG. 3).

HYPO sepsis patients may utilize/eliminate cholesterol and lipids as defense against sepsis. We analyzed circulating lipids in 271 sepsis patients and found that HYPO sepsis patients had significantly lower levels of cholesterol esters (CE, q<0.0001), lysophosphatidylcholines (LPC, q<0.0001), and sphingomyelins (SM, q=0.0005). The reduction in CEs, along with reduced total cholesterol levels in HYPO sepsis patients, implies free cholesterol is likely being eliminated from circulation (endotoxin clearance) and being utilized as substrate for steroid production, cell membranes, and other physiologic needs.

Reduced LPCs and SMs in HYPO sepsis may indicate reduced innate immunity. LPCs are a major component of oxidized LDL, and are decreased in sepsis non-survivors. LPCs protect against lethal sepsis by stimulating neutrophils to eliminate invading pathogens through an H₂O₂-dependent mechanism. SMs are a major plasma and cell membrane component. Certain bacterial toxins trigger the conversion of SMs to ceramides by sphingomyelinase, leading to localization of lysosomes and release of cathepsin B and D from lysosomes in the cytoplasm, leading to the formation of 1L-1β and TNF-α. SMs conversion to ceramides also stimulates neutrophil extracellular trap (NET) formation, which can be augmented by drugs such as tamoxifen (FDA-approved breast cancer drug). Thus, utilization and/or elimination of cholesterol and depletion of protective lipids may contribute to dysregulated metabolic and inflammatory responses in sepsis.

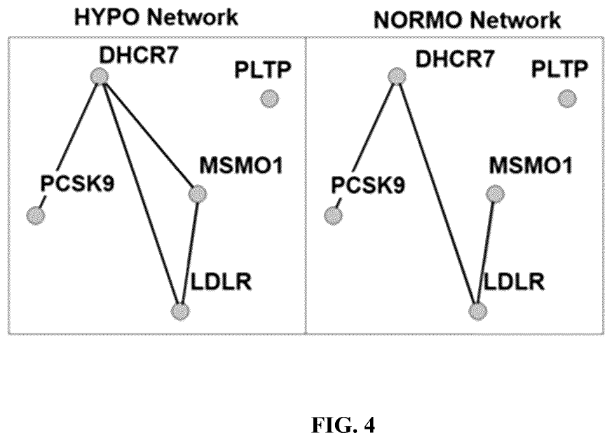

To understand causal mechanistic pathways between subphenotypes, we constructed a correlation-based network analysis (FIG. 4) that showed the major difference in HYPO sepsis vs NORMO sepsis was dependence of DHCR7 expression on MSMO1 in HYPO sepsis.

Our data indicate that 35-40% of sepsis patients have the HYPO sepsis subphenotype.

### Example 2. Upregulated Genes were Identified that Characterized the HYPO Sepsis Subphenotype

Leukocytes are important for lipid metabolism/transport in circulation. We analyzed first-24 hr. leukocyte samples from 184 UF sepsis patients (87 HYPO sepsis, 97 NORMO sepsis) using RNAseq. We interrogated 47 lipid metabolism genes, and identified 7 genes that were differentially expressed in the HYPO sepsis subphenotype (Table 2). HYPO sepsis patients had upregulation of 7 lipid metabolism genes. Q-values were adjusted p-values after false discovery rate correction. Three of the seven genes have roles in endotoxin clearance and three catalyze steps in cholesterol biosynthesis.

1. Proprotein convertase subtilisin/kexin type 9 (PCSK9) enables endotoxin clearance via the LDL receptor and promotes endothelial dysfunction via TLR4/MyD88/NF-κB and NLRP3 pathways.

2. The LDL receptor gene (LDLR) encodes the LDL receptor, which endocytoses LDL-bound endotoxin removing it from circulation.

3. Plasma phospholipid transfer protein (PLTP) transfers phospholipids from triglyceride-rich lipoproteins (VLDL, LDL) to HDL, regulating HDL's size and plays a role in endotoxin clearance and immune modulation.

4. 7-dehydrocholesterol reductase (DHCR7) catalyzes a critical step in cholesterol biosynthesis and is a precursor for vitamin D.

5. Farnesyl-diphosphate farnesyltransferase 1 (FDFT1) encodes the membrane-associated enzyme located at a branch point in the mevalonate pathway, the first enzyme in cholesterol biosynthesis, catalyzing formation of squalene.

6. Methylsterol monooxygenase 1 (MSMO1) catalyzes three-step mono-oxygenation required for the demethylation of 4,4-dimethyl and 4-alpha-methylsterols, which can be subsequently metabolized to cholesterol.

7. Arachidonate 5-lipoxygenase (ALOX5) generates 5-HETE, which leads to leukotriene production. Inhibitors of ALOX5 (5-lipooxygenase inhibitors; e.g., zileuton) are known and have been FDA approved for the treatment of asthma. Knockout of 5LOX (encoded enzyme) has been shown to protect against myocardial injury, sepsis-mediated inflammation, oxidative stress, and apoptosis (Xie S, et al. 2021).

TABLE 2
Differentially Expressed Genes in HYPO sepsis
vs. NORMO sepsis patients (RNAseq)

| gene | Fold change | logFoldChange | Q-value |
|---|---|---|---|
| PCSK9 | 4.65 | 2.2168 | 1.96 × 10⁻⁵ |
| DHCR7 | 2.00 | 1.0025 | 4.58 × 10⁻⁵ |
| LDLR | 1.80 | 0.8479 | 0.0002 |
| ALOX5 | 1.30 | 0.3820 | 0.0010 |
| PLTP | 1.44 | 0.5290 | 0.0029 |
| FDFT1 | 1.14 | 0.1864 | 0.0035 |
| MSMO1 | 1.36 | 0.4477 | 0.0046 |

### Example 3. Measurement of Gene Expression in Subject Samples

Gene expression of the 7 HYPO sepsis genes in leukocytes is determined. Whole blood is processed and leukocytes isolated. RNA is then extracted perform RT-qPCR and/or RNAseq performed to determine expression levels. RT-qPCR expression data between HYPO sepsis vs. NORMO sepsis patients (N=460) is compared. The expression data is further used to determine a HYPO sepsis risk score which can be used to discriminate between HYPO sepsis vs. NORMO sepsis.

### Example 4. SNP Idenification

Genetics can influence expression patterns and drug metabolism. Genetics can also cause or increase risk of or susceptibility to HYPO sepsis. Therefore, SNPs will be identified in the 7 HYPO sepsis genes and their effects on gene expression determined. Genotyping of the 7 key HYPO genes is performed to identify SNPs and analyze the association of each SNP with up or downregulation of all 7 HYPO sepsis genes. Associations between SNPs and up and/or downregulation of key genes and oxidized lipids, inflammatory markers, and outcomes are determined. SNPs in MSMO1 that may have a mediating effect between HYPO vs. NORMO are also evaluated.

Identify SNPs in 7 HYPO sepsis genes that alter metabolism of drugs of interest are also identified. Using PharmGKB, an NIH-funded resource that curates data on clinically actionable gene-drug associations, SNPs in 3 (of 7) HYPO sepsis genes known to affect metabolism of drugs that act on HYPO genes were identified (Table 3). The prevalence of SNPs in sepsis patients will be determined to assess potential utility to guide drug choice in future clinical trials.

TABLE 3
Drugs that inhibit HYPO genes and relevant SNPs above
that affect metabolism of the listed gene-related drug.

| Gene | SNPs | Approved Drugs | Drug mechanism |
|---|---|---|---|
| PCSK9 | rs11591147 | evolocumab | Inhibits PCSK9 decreasing LDL receptor |
| | rs11591147 | | degradation; ↑ LDL clearance |
| | rs7552841 | | |
| LDLR | rs2738466 | evolocumab | Inhibits PCSK9 decreasing LDL receptor |
| | rs14158 | | degradation; ↑ LDL clearance |
| | rs1433099 | | |
| | rs5925 | | |
| | rs688 | | |
| DHCR7 | none | cholic acid | Replaces bile acids; binds farnesoid X |
| | | | receptor regulating bile acid synthesis |
| | none | tamoxifen | Binds estrogen receptors; inhibits EBP, |
| | | | which catalyzes zymostenol to lathosterol, a |
| | | | 7DHC precurso.53 |
| ALOX5 | none | zileuton | Inhibits ALOX5 |
| | rs2115819 | montelukast | Inhibit CYSLTR1, a target of leukotrienes |

1. A method of treating or preventing HYPO sepsis in a subject, the method comprising:
(a) measuring, or having measured, expression of PCSK9, LDLR, PLTP, DHCR7, and MSMO1 in the sample obtained from the subject;
(c) determining whether expression of PCSK9, LDLR, PLTP, DHCR7, and MSMO1 is increased in the sample relative to expression of PCSK9, LDLR, PLTP, DHCR7, and MSMO1 in a predetermined control; and
(d) if expression of PCSK9, LDLR, PLTP, DHCR7, and MSMO1 is increased in the sample relative to expression in the predetermined control, then administering to the subject one or more inhibitors of one of more of PCSK9, LDLR, PLTP, DHCR7, FDFT1, MSMO1, and ALOX5.

2. The method of claim 1, wherein the method comprises:
(a) measuring, or having measured, expression of PCSK9, LDLR, PLTP, DHCR7, MSMO1, FDFT1, and ALOX5 in the sample obtained from the subject;
(c) determining whether expression of PCSK9, LDLR, PLTP, DHCR7, MSMO1, FDFT1, and ALOX5 is increased in the sample relative to expression of PCSK9, LDLR, PLTP, DHCR7, MSMO1, FDFT1, and ALOX5 in a predetermined control; and
(d) if expression of PCSK9, LDLR, PLTP, DHCR7, FDFT1, MSMO1, and ALOX5 is increased in the sample relative to expression in the predetermined control, then administering to the subject one or more inhibitors of one of more of PCSK9, LDLR, PLTP, DHCR7, FDFT1, MSMO1, and ALOX5.

3. The method of claim 1 wherein the subject has been diagnosed with sepsis, is a risk of developing sepsis, or is a risk of developing HYPO sepsis.

4. The method of claim 1, wherein treating or preventing HYPO sepsis in a subject comprises:
(a) reducing the risk of mortality associated with HYPO sepsis or increasing survival rate of the subject with or at risk of developing HYPO sepsis;
(b) preventing progression from HYPO sepsis to severe sepsis, septic shock, or chronic critical illness (CCI);
(c) preventing or decreasing the risk of multiple organ dysfunction syndrome associated with HYPO sepsis; and/or
(d) ameliorating one or more symptoms associated with HYPO sepsis, reducing the severity of HYPO sepsis, or reducing the duration of HYPO sepsis.

5. The method of claim 1, wherein measuring expression of PCSK9, LDLR, PLTP, DHCR7, and MSMO1 in the sample comprises:
(a) measuring the level of RNA one or more of PCSK9, LDLR, PLTP, DHCR7, and MSMO1 in the sample;
(b) measuring the level of one or more of PCSK9, LDLR, PLTP, DHCR7, and MSMO1 in the sample; and/or
(c) measuring activity of one or more of PCSK9, LDLR, PLTP, DHCR7, and MSMO1 in the sample.

6. The method of claim 1, wherein the expression of PCSK9, LDLR, PLTP, DHCR7, and MSMO1 is measured in leukocyte cells.

7. The method of claim 1, wherein the predetermined control comprises: a standard derived from a population of known healthy subjects.

8. The method of claim 1, wherein the one or more inhibitors are selected from the groups consisting of: evolocumab, alirocumab, bococizumab, 1D05-IgG2, RG-7652, LY3015014, inclisiran, AY9944, ziprasidone, ketanserin, pergolide, vilazodone, brexpiprazole, indacaterol, cholic acid, tamoxifen, clozapine, cariprazine, perospirone, nefazodone, aripiprazole, risperidone, metoprolol, fluoxetine, trazodone, haloperidol, ifenprodil, cyclopamine, doxorubicin, BM 15.766, and GANT61, ER-28488, ER-27856, RPR 107393, YM-53601, schizostatin, zaragozic acid A, benzoxazepines, squalene synthase inhibitor, bavachinin, zileuton, montelukast, meclofenamate sodium, myxochelin, and nordihydroguaiaretic acid.

9. A method of treating sepsis in a subject comprising:
(a) identifying whether the subject is at risk of HYPO sepsis by
(i) measuring levels of PCSK9, LDLR, PLTP, DHCR7, and MSMO1 in a sample obtained from the subject;
wherein levels of each of PCSK9, LDLR, PLTP, DHCR7, and MSMO1, in the sample greater than a predetermined control indicates the subject has or is a risk of developing HYPO sepsis; and
(b) administering to the subject identified as having HYPO sepsis or being at risk of developing HYPO sepsis one or more inhibitors of one of more of PCSK9, LDLR, PLTP, DHCR7, FDFT1, MSMO1, and ALOX5.

10. The method of claim 9, wherein measuring expression of PCSK9, LDLR, PLTP, DHCR7, and MSMO1 in the sample comprises:
(a) measuring the level of RNA one or more of PCSK9, LDLR, PLTP, DHCR7, and MSMO1 in the sample;
(b) measuring the level of one or more of PCSK9, LDLR, PLTP, DHCR7, and MSMO1 in the sample; and/or
(c) measuring activity of one or more of PCSK9, LDLR, PLTP, DHCR7, and MSMO1 in the sample.

11. The method of claim 9, wherein the method comprises:
(a) identifying whether the subject is at risk of HYPO sepsis by
(i) measuring levels of PCSK9, LDLR, PLTP, DHCR7, FDFT1, MSMO1, and ALOX5 in a sample obtained from the subject;
wherein levels of each of PCSK9, LDLR, PLTP, DHCR7, FDFT1, MSMO1, and ALOX5 in the sample greater than a predetermined control indicates the subject has or is a risk of developing HYPO sepsis; and
(b) administering to the subject identified as having HYPO sepsis or being at risk of developing HYPO sepsis one or more inhibitors of one of more of PCSK9, LDLR, PLTP, DHCR7, FDFT1, MSMO1, and ALOX5.

12. The method of claim 9, wherein the expression of PCSK9, LDLR, PLTP, DHCR7, and MSMO1 is measured in leukocyte cells.

13. The method of claim 9, wherein the predetermined control comprises: a standard derived from a population of known healthy subjects.

14. The method of claim 9, wherein the one or more inhibitors are selected from the groups consisting of: evolocumab, alirocumab, bococizumab, 1D05-IgG2, RG-7652, LY3015014, inclisiran, AY9944, ziprasidone, ketanserin, pergolide, vilazodone, brexpiprazole, indacaterol, cholic acid, tamoxifen, clozapine, cariprazine, perospirone, nefazodone, aripiprazole, risperidone, metoprolol, fluoxetine, trazodone, haloperidol, ifenprodil, cyclopamine, doxorubicin, BM 15.766, and GANT61, ER-28488, ER-27856, RPR 107393, YM-53601, schizostatin, zaragozic acid A, benzoxazepines, squalene synthase inhibitor, bavachinin, zileuton, montelukast, meclofenamate sodium, myxochelin, and nordihydroguaiaretic acid.

15. A method for treating or preventing HYPO sepsis in a subject, the method comprising:
(a) measuring, in a sample obtained the subject, levels of PCSK9, LDLR, PLTP, DHCR7, and MSMO1 expression in the sample;
(c) correlating the levels of PCSK9, LDLR, PLTP, DHCR7, and MSMO1 expression in the sample with a reference level obtained from a predetermined control or standard derived from a population of healthy subjects to determine whether the subject has or is at risk of developing HYPO sepsis; and
(d) if the levels of expression of each of PCSK9, LDLR, PLTP, DHCR7, and MSMO1 is greater than the reference level, then administering at least one pharmaceutically effective dose of one or more inhibitors of one of more of PCSK9, LDLR, PLTP, DHCR7, FDFT1, MSMO1, and ALOX5.

16. The method of claim 15, wherein measuring expression of PCSK9, LDLR, PLTP, DHCR7, and MSMO1 in the sample comprises:
(a) measuring the level of RNA one or more of PCSK9, LDLR, PLTP, DHCR7, and MSMO1 in the sample;
(b) measuring the level of one or more of PCSK9, LDLR, PLTP, DHCR7, and MSMO1 in the sample; and/or
(c) measuring activity of one or more of PCSK9, LDLR, PLTP, DHCR7, and MSMO1 in the sample.

17. The method of claim 15, wherein the method comprises:
(a) measuring, in a sample obtained the subject, levels of PCSK9, LDLR, PLTP, DHCR7, FDFT1, MSMO1, and ALOX5 expression in the sample;
(c) correlating the levels of PCSK9, LDLR, PLTP, DHCR7, FDFT1, MSMO1, and ALOX5 expression in the sample with a reference level obtained from a predetermined control or standard derived from a population of healthy subjects to determine whether the subject has or is at risk of developing HYPO sepsis; and
(d) if the levels of expression of each of PCSK9, LDLR, PLTP, DHCR7, FDFT1, MSMO1, and ALOX5 is greater than the reference level, then administering at least one pharmaceutically effective dose of one or more inhibitors of one of more of PCSK9, LDLR, PLTP, DHCR7, FDFT1, MSMO1, and ALOX5.

18. The method of claim 15, wherein the sample comprises leukocyte cells.

19. The method of claim 15, wherein the predetermined control comprises: a standard derived from a population of known healthy subjects.

20. The method of claim 15, wherein the one or more inhibitors are selected from the groups consisting of: evolocumab, alirocumab, bococizumab, 1D05-IgG2, RG-7652, LY3015014, inclisiran, AY9944, ziprasidone, ketanserin, pergolide, vilazodone, brexpiprazole, indacaterol, cholic acid, tamoxifen, clozapine, cariprazine, perospirone, nefazodone, aripiprazole, risperidone, metoprolol, fluoxetine, trazodone, haloperidol, ifenprodil, cyclopamine, doxorubicin, BM 15.766, and GANT61, ER-28488, ER-27856, RPR 107393, YM-53601, schizostatin, zaragozic acid A, benzoxazepines, squalene synthase inhibitor, bavachinin, zileuton, montelukast, meclofenamate sodium, myxochelin, and nordihydroguaiaretic acid.
